(19) United States
(12) Patent Application Publication
(10) Pub. No.: US 2019/0392925 A1
(43) Pub. Date: Dec. 26, 2019

(54) SELF-AWARE DATA STORAGE, RETRIEVAL, AND NOTIFICATION

(71) Applicant: Group Healthcare Limited, Boise, ID (US)

(72) Inventors: Peter James Wachtell, Boise, ID (US); Sam Jacobs, Dairy Flat (NZ)

(21) Appl. No.: 16/015,271

(22) Filed: Jun. 22, 2018

(51) Int. Cl. G16H 10/60; G06F 17/30; G06F 7/14; G06F 21/62

(52) U.S. Cl. CPC G16H 10/60; G06F 17/30241; G06F 8/61; G06F 7/14; G06F 21/6227; G06F 17/30312

(57) ABSTRACT

Techniques for providing and operating a database system are disclosed. The techniques can include providing client software for installation at each of a plurality of disparate database systems; receiving, by a computer server, indexes provided by the client software; merging, by the computer server, data from the indexes to produce a master index; identifying patients that have upcoming appointments or recent changes to their clinical facts; for each of the identified patients, building in transient electronic memory a respective representation of encoded clinical facts from multiple of the plurality of geographically disparate database computer systems using the master index; comparing each respective representation of encoded clinical facts to each of a plurality of clinical event templates to detect at least one match; and for at least one match, providing a notification to a care provider of a respective patient.

### FIELD

Disclosed techniques relate to database management and usage.

### BACKGROUND

In many circumstances, data concerning a single topic may be spread across many separate databases. For example, in the case of patient medical records, data about each patient may be present in many disparate databases. Further, data in databases is generally static and provides no information as to its relevancy to other data that may be in separate databases. Data in a database may be neither linked nor correlated to other relevant data. In the case of medical records, patient harm can occur when uncorrelated data is not cross referenced by the appropriate care providers. For example, a prescription issued by one doctor and may be filled for a patient who has been diagnosed with a relevant disease (for which that specific medication is contraindicated) by a second doctor, resulting in the issuance of a prescription that can cause harm.

Prior attempts to solve for this kind of non-correlation have involved creating a universal medical record, with information about the patient from various discrete databases pulled into a single dataset. Such a single dataset can be sorted, filtered, and acted upon to identify when a patient is on a path to potential harm. Such methods have proven to be costly, subject to considerable political, legal and ethical challenges, difficult to execute, vulnerable to hacking, and hard to apply over a large geographic area and across large numbers of discrete databases.

In the United Kingdom, billions of British pounds have been spent pursuing a solution to this problem with limited results and a conclusion by the medical administrators that their attempt to achieve a universal medical record has resulted in failure, with little to show in return for the cost and effort.

### SUMMARY

According to various embodiments, a computer implemented method is presented. The method includes providing client software for installation at each of a plurality of disparate database systems, where the client software includes computer readable instructions which, when executed by a client computer, cause the client computer to provide to a computer server over a computer network a respective index at least upon changes to any patient record in a respective database; receiving, by a computer server and over the computer network, indexes provided by the client software installed on at least some the plurality of geographically disparate database systems, where each index includes data encoding at least one patient and at least one associated clinical fact stored at a respective database; merging, by the computer server, data from the indexes provided by the client software installed on each of the plurality of geographically disparate database systems to produce a master index; identifying a plurality of patients that have upcoming appointments or recent changes to their clinical facts; for each of the plurality of patients, building in transient electronic memory a respective representation of encoded clinical facts from multiple of the plurality of geographically disparate database computer systems using the master index; comparing each respective representation of encoded clinical facts to each of a plurality of clinical event templates to detect at least one match; and for at least one match, providing a notification to a care provider of a respective patient.

Various optional features of the above embodiments include the following. In some embodiments, no clinical records leave their respective database of the plurality of disparate database computer systems. The encoded clinical facts may include encrypted clinical facts. The plurality of disparate database computer systems may include a plurality of geographically disparate database computer systems, a plurality of legally disparate database computer systems, or a plurality of technologically disparate computer systems. The identifying may include: identifying by at least one of the plurality of disparate database computer systems; and providing corresponding identifying data to the computer server. The method may include providing, by the computer server, the master index to at least one of the plurality of disparate database computer systems. The building in transient electronic memory a respective representation of encoded clinical facts may be performed by least one of the plurality of disparate database computer systems. The client software may further include computer readable instructions which, when executed by a client computer, cause the client computer to translate changes to patient records into encoded clinical facts. The notification may include at least one electronic link to a clinical record of a respective patient in a respective one of the plurality of geographically disparate database computer systems. The method may include, prior to the providing a notification, receiving authorization from the respective one of the plurality of geographically disparate database computer systems to provide the link.

According to various embodiments, a computer system is provided. The computer system includes non-transitory computer-executable client software installed on a plurality of disparate database computer systems communicatively coupled to a computer network, where the client software includes computer readable instructions which, when executed by a respective client computer, cause the respective client computer to provide to a computer server a respective index at least upon changes to any patient record in a respective database; and a computer server computer communicatively coupled to the computer network, where the computer server is configured to: receive indexes provided by the client software installed on at least some of the plurality of geographically disparate database systems, where each index includes data encoding at least one patient and at least one associated clinical fact stored at a respective database, and merge data from the indexes provided by the client software installed on each of the plurality of geographically disparate database systems to produce a master index; where the system is further configured to: identify a plurality of patients that have upcoming appointments or recent changes to their clinical facts; for each of the plurality of patients, build in transient electronic memory a respective representation of encoded clinical facts from multiple of the plurality of geographically disparate database computer systems using the master index; compare each respective representation of encoded clinical facts to each of a plurality of clinical event templates to detect at least one match; and for at least one match, provide a notification to a care provider of a respective patient.

Various optional features of the above embodiments include the following. According to some embodiments, no clinical records leave their respective database of the plurality of disparate database computer systems during operation of the system. The encoded clinical facts may include encrypted clinical facts. The plurality of disparate database computer systems may include a plurality of geographically disparate database computer systems, a plurality of legally disparate database computer systems, or a plurality of technologically disparate computer systems. The system may be further configured to identify a plurality of patients that have upcoming appointments or recent changes to their clinical facts by: identifying, by at least one of the plurality of disparate database computer systems, a plurality of patients that have upcoming appointments or recent changes to their clinical facts; and providing, by at least one of the plurality of disparate database computer systems, corresponding identifying data to the computer server. The computer server may be further configured to provide the master index to at least one of the plurality of disparate database computer systems. The system may be further configured to build in transient electronic memory of at least one of the plurality of disparate database computer systems a respective representation of encoded clinical facts. The client software may further include computer readable instructions which, when executed by a client computer, cause the client computer to translate changes to patient records into encoded clinical facts. The notification may include at least one electronic link to a clinical record of a respective patient in a respective one of the plurality of geographically disparate database computer systems. The system may be further configured to, prior to providing a notification, receive authorization from the respective one of the plurality of geographically disparate database computer systems to provide the link.

### DESCRIPTION OF DRAWINGS

The accompanying drawings, which are incorporated in and constitute a part of this specification, illustrate implementations of the described technology. In the figures:

FIG. 1 is a schematic diagram of system according to various embodiments;

FIG. 2 is a flow diagram of a method according to various embodiments; and

FIG. 3 is a detailed schematic diagram of a server computer system with example connections according to various embodiments.

### DETAILED DESCRIPTION

Reference will now be made in detail to the disclosed embodiments, which are illustrated in the accompanying drawings. Wherever possible, the same reference numbers will be used throughout the drawings to refer to the same or like parts. In the following description, reference is made to the accompanying drawings that form a part thereof, and in which is shown by way of illustration specific examples. These examples are described in sufficient detail to enable those skilled in the art to practice them and it is to be understood that other examples may be utilized and that changes may be made without departing from the scope of the disclosure. The following description is, therefore, merely exemplary.

Some embodiments solve the problem of disparate databases by maintaining the disparate databases, but, in a manner of speaking, making the data itself sentient and aware of when and where it may be relevant to identifying potential patient harm. This allows the databases to self-identify to the system, other databases, and the system administrator when data elements their local memories are indicative of potential harm and can be considered for triggering mitigating actions to prevent patient harm.

Some embodiments include client software run adjacent to each disparate database system. Each such discrete disparate database system can be considered a node in the overall system once configured by the client software. The client software, among other things, administers system interactions and provides to a server computer an index of patient medical records, e.g., by patient and/or by the number and type of records held in that particular local database for that patient.

According to some embodiments, the nodes communicate indexes from the various disparate database systems to the server computer, where all such indexes are collected, merged, and cross-referenced into a master index. By building such a master index, the server computer consolidates information from each node, without copying any actual data from any node. Thus, any particular node in possession of such a master index has information about all other nodes that possess data about the patients that the data in its local database references, as well and the type and number of data points that each of the other nodes contain about that patient. In other words, each node has information about where all of the other data in the system (for each patient that it holds records for locally) can be found and what type of data is represented at each correlating node. Nodes may store patient data including, by way of non-limiting example, gender, age, diagnosis, prescribed drugs, drug histories, lab results, dates of medical visits over some interval (e.g., the last 10 years), etc.

Once the server computer has generated the master index, the identification and mitigation of potential harm can occur, by way of non-limiting example, as follows. Upon the arrival of any change in any data element held at any node, the node can inform the server computer that a specific change in the data that it holds has occurred and can request instructions. The server computer can look at the data element change and can reference a table of valued process flows, for example, medical harm process flows, in which specific queries associated with such a data element change can be run against the specific index information of that patient in each node that has records that are relevant to that specific query and for that particular patient. An example of a triggering data element change is a newly issued prescription by a doctor. The relevant queries may include queries regarding the previous issuance of any of a number of contraindicated prescriptions for that patient, as well as other disease diagnosis that may indicate potential harm in conjunction with the newly prescribed drug. Additional queries may be involve, for example, calculating the trending change in the lab results from the patient, resulting in warnings as negatively trending labs may indicate that further review by a doctor is appropriate before the newly issued prescription is fulfilled at a pharmacy and taken by the patient. If a data element change is reported to the server computer and the medical harm process flow queries are run on all of the relevant nodes that hold data relevant to the specific query and the specific patient and the results returned across all nodes indicate that further review by a doctor is required, a notification may be sent to the appropriate doctor prompting for a review of the pertinent information. each node that holds data relevant to that specific patient and that specific harm process flow can be identified and the respective data (e.g., only the relevant data) made available to a treating clinician who is authorized to view such data. Other, non-relevant patient data remains confidential and is not accessed or viewed as it is not necessary to treat the patient.

According to some embodiments, at no point does the server computer possess personally identifying information for any specific patient, copy data elements from any of the disparate database systems, pull patient records from any of the disparate database systems, create any archive of personal medical records of any patient, or otherwise intrude upon the medical privacy of the patients whose records are stored at any of the from any of the disparate database systems. According to some embodiments, at no point is the data in any database copied, transmitted, or synced between databases or tables. According to some embodiments, at no point is the data in any database pre-authorized to be shared, placed outside of the immediate control and security of the current holder, aggregated into a single place that makes an attractive target for a hacker, or exposed to new users that may not be authorized by the data element's owner.

According to various embodiments, the server computer may hold high value process flows and the relevant questions, and these may be downloaded to the relevant node when it indicates that is has received a data element change. The determination of relevance may occur at the local node, and the node itself may determine the ensuing actions such as contacting an authority for further review and actions surrounding the sharing of the relevant data.

Embodiments may be utilized in a variety of settings. Embodiments may be utilized by, for example, any large medical company or community or an entity that deals with multiple patients with relevant patient data spread across multiple discrete datasets that are owned and controlled by differing authorities or are regulated by competing regulatory structures. Further, although the present description proceeds with a detailed description primarily in terms of a medical setting, embodiments are not so limited. Embodiments have applicability in other value-producing process flows that are dependent upon or enhanced by the correlation of data held in discrete separate databases with ownership or access/regulatory authority that is diverse or dispersed. In general, embodiments may allow a diverse set of data regulators to permit the data to be used for processes only in those circumstances where such data is actually relevant and value producing. Additional possible settings for embodiments are described far below, as well as how such embodiments differ from the medical setting embodiments that are described presently.

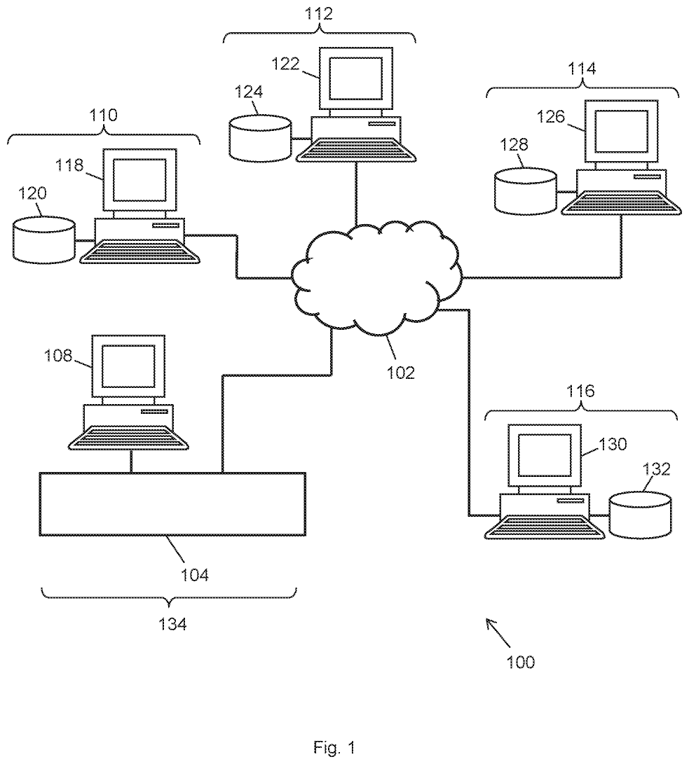

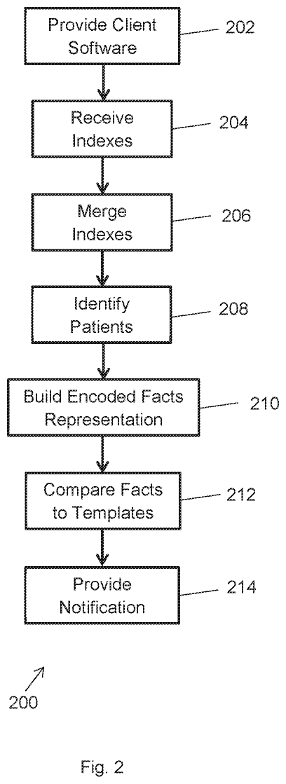

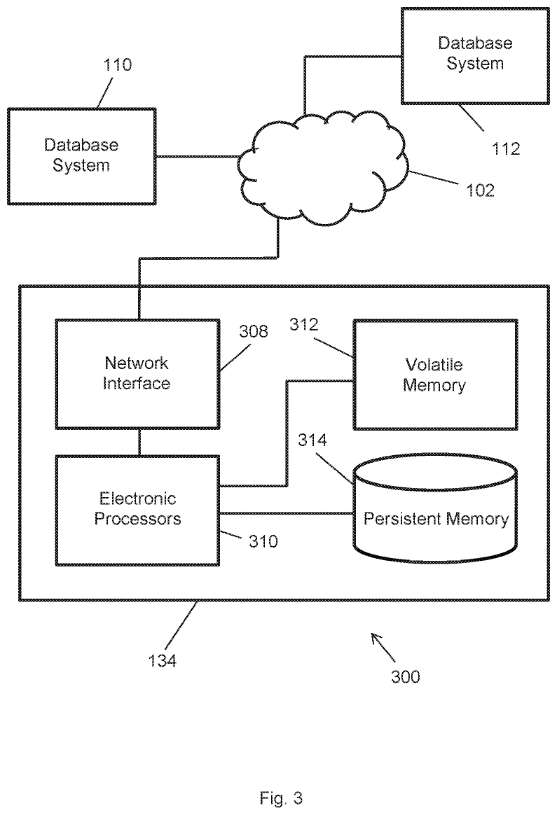

FIG. 1 is a schematic diagram of system 100 according to various embodiments. System 100 includes server computer 104, which is communicatively coupled to network 102 such as the internet, and which may be coupled to an administrative terminal 108 to form server computer system 134. Server computer 104 stores updated rules for dissemination to each database system 108, 110, 112, 114, described further below. Server computer 104 may coordinate event notifications when events are detected by one or more database system 108, 110, 112, 114. The functionality of server computer 104 is shown and described in detail in reference to FIG. 2, and the internal architecture of server computer 104 is shown and described in detail in reference to FIG. 3.

System 100 also includes a plurality of disparate database systems 110, 112, 114, 116, each communicatively coupled to network 102. Each database system 110, 112, 114, 116, includes a respective terminal 118, 122, 126, 130, through which a local administrator may configure and supervise its operation. Each database system 110, 112, 114, 116, includes some form of documented access protocol (API, or manual) through which they may interact with server computer 104, for example. Each database system 110, 112, 114, 116, includes, or is communicatively coupled to, a respective persistent storage 120, 124, 128, 132.

Persistent storage 120, 124, 128, 130 stores both medical data for a plurality of patients, although typically not the same set of patients in any two such persistent storage devices. Persistent storage 120, 124, 128, 130 may include a traditional form of formatted electronic data storage, such as tables or some documented data structure. That is, persistent storage 120, 124, 128, 130 stores a patient database and/or database tables.

Persistent storage 120, 124, 128, 130 also stores a client software instance in computer-executable non-transitory format. The client software facilitates communications with server computer 104 as described herein, particularly in reference to FIG. 2, below. Once installed in a respective database system 110, 112, 114, 116, such database system operates as a node and can communicate with server computer 104 as described herein. The client software may be a thin client that configures each of database systems 110, 112, 114, 116 as a node according to some embodiments. The client software may be coded in any suitable computer language and run in any suitable operating system. The client software may execute on a respective database systems 110, 112, 114, 116 adjacent to the stored data.

Note that each of database systems 110, 112, 114, 116, as well as server computer system 134, may be owned, controlled, operated, and/or administered by different entities. Further, each of database systems 110, 112, 114, 116 and server computer system 134 may be geographically present anywhere, and coupled to any electronic communication channel. Thus, database systems 110, 112, 114, 116 may be any of physically, legally, geographically, or administratively disparate.

FIG. 2 is a flow diagram of a method 200 according to various embodiments. Method 200 is presented from the perspective of a server computer, such as server computer 104, interacting with nodes, such as database systems 110, 112, 114, 116 properly configured with instances of the small computer program described herein. Method 200 may be implemented in part by database systems, such as database systems 110, 112, 114, 116, together with a server computer, such as server computer 104. For purposes of illustration rather than limitation, method 200 is described in reference to system 100 of FIG. 1.

At block 202, client software is provided to each of database systems 110, 112, 114, 116. The client software may be provided to database systems 110, 112, 114, 116 via a variety of communication channels, including, for example, the internet or other network, persistent non-transitory computer readable media sent through the mail or other form of physical transport, or other types of channels. A respective administrator of each of database systems 110, 112, 114, 116 may obtain, install, and execute the client software in his or her respective database system.

At block 204, server computer 104 receives indexes generated by one or more of database systems 110, 112, 114, 116. Server computer 104 may receive such indexes via network 102, for example. Each index includes encoded representations of the patients whose medical data is stored at the respective database system 110, 112, 114, 116, as well as encoded related medical facts regarding such patients. The encoding of the patient representation and related medical facts may be encrypting according to some embodiments. According to other embodiments, the encoding may include using representative strings of alphanumeric characters instead of recorded medical facts, where the alphanumeric character strings are not readily interpretable as the encoded facts without extra information such as a translation table correlating codes with their represented medical facts. Note that any of database systems 110, 112, 114, 116, as well as server computer 104, may store one or more such translation tables in persistent storage, for example. Alternatively, a dynamic encoding key may be used for reading or translating the data into a readable form. Such embodiments may utilize hashing with reading occurring with the use of a hash key, which may be stored or which may be derived by an algorithm created using aspects of the system architecture.

Each database system 110, 112, 114, 116 may generate and send to server computer 104 a respective index at various times, and due to various triggering events, as explained presently. According to some embodiments, the client software in a database system 110, 112, 114, 116 detects whenever a change is made to any of its stored patient data. Such a change may include the addition or deletion of data, for example. Upon such detection, the client software generates an index for all patients and the patients' medical facts stored in its database. According to some embodiments, the client software in a database system 110, 112, 114, 116 detects patients for whom a doctor's appointment is upcoming and generates an index for the patients in the respective database. The former detection, regarding patient data changes, as well as the sending of the generated indexes to server computer 104, may occur periodically (e.g., every ten minutes) or substantially in real time as data changed occur. The latter detection, regarding patients with upcoming appointments, as well as the sending of the generated indexes to server computer 104, may occur periodically, e.g., every morning before the start of business, and encompass patients that have an appointment scheduled for later that day. Further, the latter detection may be performed in batches. Note that for both the former and the latter detection, a corresponding index is generated and pushed to server computer 104 by database systems 110, 112, 114, 116 without requiring any actions from server computer 104. In at least this sense, the nodes of database systems 110, 112, 114, 116 are active and monitoring their own data and, when combined with interactions with the server, become sentient in a sense regarding the relevancy of the data that is held in the node to some aspect of the patient's clinical needs.

Each database system 110, 112, 114, 116 may generate its respective index as follows. The client software at each database system 110, 112, 114, 116 that provides such an index may access stored patient data concerning patients with record changes or upcoming appointments, for example, using an API, through direct access, or a via pseudo-manual access processes (e.g., ghost typing). The data may be mapped (e.g., identified according to storage in a particular database system) and indexed by person, type of data elements held, number of data elements held, and/or dates of data element origination. Each index may be stored at its respective database system 110, 112, 114, 116, before being sent to server computer 104.

Note that the indexes may be of any of a variety of forms. According to some embodiments, each index relates to a single patient and encodes facts relative to that patient. For such embodiments, each respective database system 110, 112, 114, 116 may include multiple indexes for each patient. According to some such embodiments, each index encodes facts related to a single aspect of a single patient, e.g., an index of vaccination records for Patient X, a separate index for prescriptions for Patient X, another index for diagnoses for Patient X, etc. According to other such embodiments, each index encodes multiple dimensions of data for a patient, and according to other embodiments, each index is a comprehensive repository of medical data for a patient. According to other embodiments, each index encodes medical facts for more than one patient. According to some such embodiments, each index encodes facts related to a single aspect of multiple patients. According to other such embodiments, each index encodes multiple dimensions of data for each of multiple patients. According to such embodiments, each respective database system 110, 112, 114, 116 may store one or more index for each patient.

Note that the term “index” relates to each index being capable of organized according to the values of one (or more, e.g., lexicographically) variable at a time. Some embodiments utilize a high-level, e.g., object oriented, programming language that describes each patient as a single object with multiple attributes (name, age, disease code). Other embodiments utilize a flat file for each index, where the flat file encodes the relevant data. For such embodiments, the flat files may be processed using computer-executable code, e.g., a low-level language, to extract the index, that is, the ordered arrangement of data. Data structures with complexity intermediate between flat files and high-level objects are also possible.

Upon receiving the indexes from database systems 110, 112, 114, 116, server computer 104 holds them in volatile memory without storing them in persistent memory. The volatile memory may include one or more Random Access Memory (“RAM”) physical devices, for example.

At block 206, server computer 104 merges the respective indexes received from any of database system 110, 112, 114, 116 into a cohesive master index. The merging of the indexes into a master index may include holding them in volatile memory and copying their data to a single table structure, for example, which may also be held in volatile memory, without any index data being stored to persistent memory according to some embodiments. The master index provides a mapping of data for each patient, what types of data elements are held, how many elements there are, their origination dates as well as any edit dates, and where they can be found.

The master file may have any of a variety of forms and may be built from the respective indexes received from database systems 110, 112, 114, 116 in a variety of manners. According to some embodiments, the master index includes is a flat file that includes encoded representations of all of the relevant patients and associated attributes. Computer executable code may be used to extract the index information therefrom, according to some embodiments. According to other embodiments, the master index is an object in a high-level object oriented programming language. The master index can be indexed by any of the attributes, or by a combination of attributes, e.g., to produce index of males, over 40, with diabetes, organized lexicographically by age, then by date of diabetes diagnosis.

Note that according to various embodiments, the master index may include data as organized, presented, or otherwise included in the constituent indexes in a manner similar to that of the indexes, or in a different manner. Thus, according to some embodiments, the master index includes the same data, arranged in a similar manner, as the various indexes from which it is built. According to other embodiments, the master index includes data in addition to the data received from database systems 110, 112, 114, 116. Such additional data may be data that is not accessible to any of database systems 110, 112, 114, 116. For example, such additional data may include distance of the practice represented by the respective database systems 110, 112, 114, 116 to a lab or to a hospital, or distance from that patient's address to a pharmacy. Additional forms of data can include, e.g., inputs from national, regional, or local databases, such as a list of children who have received various vaccinations, or women who have received mammograms. Thus, server computer 104 may have relevant new data elements with additional attributes (and indexing based on those attributes). Server computer 104 may push such added data down to the database systems 110, 112, 114, 116 for inclusion in their respective indexes.

At block 208, server computer 104 identifies certain patients for analysis of the potential for a medical adverse event. The patients may be identified by determining patients with recent changes to their stored patient data and patients with upcoming appointments (e.g., that day). Such changes to patient data may include the addition or deletion of data. Note that according to some embodiments, the identification of this block may utilize identification information as determined as part of the actions of block 204. According to other embodiments, the identification of this block can utilize the detection processes described above in reference to block 204.

At block 210, server computer 104 builds in transient electronic memory, for each patient identified per block 208, a respective representation of encoded clinical facts about the patient. Server computer 104 builds each encoded representation of encoded clinical facts using the master index. In particular, for each identified patient, server computer 104 searches the master index for any data representing information for that patient. Server computer 104 assembles any information detected by the search into a comprehensive in-memory encoded record for the patient, for each patient identified at block 208. The format of the assembled information may be any of a variety of types, not limited to eXtensible Markup Language (“XML”) or a database table structure. Thus, at the end of the actions for this block, server computer 104 holds in memory one or more comprehensive representations of encoded clinical facts about respective one or more patients.

At block 212, server computer 104 compares each of the in-memory comprehensive representations of encoded clinical facts to each of a plurality of clinical event templates. Each clinical event template specifies codes for a suite of clinical facts that, when present in records for a patient, indicate the possibility of a medical adverse event for the patient. The templates may be obtained from a variety of sources. For some embodiments, they are created with the assistance of clinical authorities. The clinical codes in the templates represent things like someone getting a prescription or change in prescription for blood pressure medication while seeing a prescription out there for Viagra. These two are not compatible, but may have been written by different doctors due to the patient's embarrassment. Another example is a template that includes clinmical codes that represent the following situation: a very normal, safe drug like allopurinol being prescribed for gout when there is an old lab report from a hospital stay year prior in which kidney damage is indicated. The templates may be in any of a variety of formats, not limited to XML and database table format. For embodiments that store the comprehensive representations of encoded clinical facts in database table format, the templates may be used to generate database queries (e.g., SQL queries), which may then be made to the in-memory comprehensive representations of encoded clinical facts. An example of pseudocode for a query representing a determination of whether the patient is 18-75 years of age with diabetes and has hemoglobin A1c>64 is as follows: “pat_ageY between 15 and 74 and diabmel_date is not null and hba1c_value>64”. If such a determination is met, then the patient is at risk for the adverse medical event of “Hemoglobin A1c (HbA1c) Poor Control”. Some embodiments have one template per potential adverse medical event; other embodiments may have more than one event represented per template (or multiple templates representing a single event).

Note that server computer 104 may utilize one or more stored translation tables that correlate codes as present in the encoded clinical facts to their represented medical facts. Server computer 104 may utilize such translation tables to ensure that any matches between encoded clinical facts present in the comprehensive representations and clinical facts as represented in the templates are detected. Further, server computer 104 may utilize such translation tables to merge the various indexes at block 206.

The comparing at this block determines whether the facts of any of the clinical event templates match (e.g., are the same as) the facts represented in any of the patients' in-memory comprehensive representations of encoded clinical facts. If so, then method 200 proceeds to block 214. Otherwise, method 200 may terminate.

At block 214, server computer provides a notification to a care provider of any patient for which a match is detected at block 212. The notifications may be sent via any of a variety of communication channels. According to various embodiments, server computer sends the notifications via email, text message, automated phone call, or messaging application. The notifications may be sent to care providers at any medical facility that stores records at any of database systems 110, 112, 114, 116. Alternately, or in addition, the notifications may be sent to a primary care physician. The notifications may include hyperlinks or other types of links to relevant patient records at any of database systems 110, 112, 114, 116. In such embodiments, if a receiving entity is authorized to view records in a respective database system, then the entity may use such links to do so; otherwise, the entity may not view the linked records.

This concludes the description of method 200 for embodiments that employ a central server, such as server computer 104, to handle most of the tasks.

Other embodiments may utilize peer-to-peer techniques that rely less on any central server. Differences between the above-described embodiments of method 200 an example peer-to-peer embodiments are described presently. In general, the actions of blocks 208, 210, 212, and 214 in such embodiments may be performed by the nodes of database systems 110, 112, 114, 116 instead of by server computer 104. In particular, according to some peer-to-peer embodiments, after server computer 104 merges the indexes to form a master index per block 206, it distributes the master index to the nodes of each database system 110, 112, 114, 116. According to such embodiments, server computer may also distribute copies of its clinical event templates to the nodes of each database system 110, 112, 114, 116. In such embodiments, the actions of blocks 210 and 212 may occur in the individual nodes. Per block 210, each node may build in memory a representation of encoded clinical facts about the patients with data in their respective database systems 110, 112, 114, 116. Per block 212, each node may identify only the subset of patients that have changed clinical data or upcoming appointments whose data is held locally. Further, in such embodiments, the nodes may communicate the identified patients and the changed data to server computer 104, which may update the master index and redistribute it to the nodes. According to such embodiments, either a respective node of database systems 110, 112, 114, 116, or server computer 104, may perform the actions of block 214. For peer-to-peer embodiments, each node of database systems 110, 112, 114, 116 may also provide the alerts to the other nodes.

FIG. 3 is a detailed schematic diagram of a server computer system 134 with example connections according to various embodiments. Server computer system 134 is communicatively coupled via network 102 to database systems 110, 112, 114, and 116, as shown and described in reference to FIG. 1, and as partially shown in reference to FIG. 3. Server computer system 134 includes server computer 104 and administrative terminal 108 as shown and described above in reference to FIG. 1. Server computer system 134 further includes internal features as shown in FIG. 3 and described presently. Server computer 104 includes network interface 308, which may include multiple server computers configured to handle massive amounts of network traffic. Server computer system 134 further includes one or more electronic processors 310, which may be configured by computer-executable non-transitive program instructions stored in persistent memory 314 to at least partially perform the methods disclosed herein, e.g., method 200 of FIG. 2. Further, server computer system 134 includes volatile memory 312, which is configured to hold the indexes received form the nodes, the master index, and the comprehensive representations of encoded clinical facts for one or more patients, as described above.

Although embodiments have been described with an example focus on patient medical records, embodiments are not so limited. For example, some embodiments may be applied to customers and purchasing activities or even to generic “things” such as vehicles and current or trending locations. Some embodiments may extend the examples to clinical notifications of patients at risk of imminent harm due to medical conditions and treatments to include external risks. For example, a government entity that performs background checks for gun purchases may have difficulty gaining full access to the medical records (e.g., mental health, diagnosis and prescriptions) of a gun acquisition applicant due to the restriction of other laws regarding access to private medical records. Embodiments may handle such a situation by including a clinical event template identifying that specify a relevant series of facts (e.g., diagnosis of depression, suicide risk, domestic anger management issues). Such an embodiment may send an alert to the government to determine if a gun purchase approval is warranted or if further review and consideration is advised.

Further, in some embodiments, the patient (or applicant, per the above example) is asked if they consent to have their record(s) viewed by the care provider (or government entity). In such embodiments, the record(s) are only viewed upon full consent of the patient/purchaser, e.g., after they have been made fully aware of the extent of records to be shared and the purpose for which such consent to access the data will be used.

Yet further embodiments may allow the sharing of data between large systems. A specific example is the sharing of intelligence data between the agencies of different countries. A very high level of trust is required for a country to grant full access to their data for use by another country. An example of the “five eyes” arrangement in which the USA, UK, Australia, New Zealand, and Canada all agree to share access to intelligence data. Germany is also a good ally of the USA, but not trusted enough to make it into the highly restricted club in which all participants received unfettered access to all data held by the group. However, if a certain set of facts were applicable to a given person, and these facts held in a master index with an event template that was agreed upon by the five eyes and Germany, full sharing of those specific records may be implemented without further human intervention using an embodiment. Note that the rapid sharing of specific data when new facts arrive about a subject is a highly demanded function in intelligence sharing where speed to reaction is of paramount importance. Such changed facts may trigger an event that grants full (or greater) access to the specific data to a specific group of users, e.g., in Germany.

Thus, a system has been presented that performs by, instead of granting a wide authority to access and view multiple datasets in preparation for the relatively few and discrete occurrences where correlated data would lead to a value producing result, the system works to identifying those situations where a value producing result exists and then asks the owners or regulators of the datasets involved for specific limited authority to access and view the limited records that are relevant to a specific identified value process.

In general, systems capable of performing the presented techniques may take many different forms. Further, the functionality of one portion of the system may be substituted into another portion of the system. Each hardware component may include one or more processors coupled to random access memory operating under control of, or in conjunction with, an operating system. The system can include network interfaces to connect with clients through a network. Such interfaces can include one or more servers. Appropriate networks include the internet, as well as smaller networks such as wide area networks (WAN) and local area networks (LAN). Networks internal to businesses or enterprises are also contemplated Further, each hardware component can include persistent storage, such as a hard drive or drive array, which can store program instructions to perform the techniques presented herein.

The foregoing description is illustrative, and variations in configuration and implementation are possible. For example, resources described as singular can be plural, and resources described as integrated can be distributed. Further, resources described as multiple or distributed can be combined. The scope of the presented techniques is accordingly intended to be limited only by the following claims.

What is claimed is:

1. A computer implemented method, the method comprising:
providing client software for installation at each of a plurality of disparate database systems, wherein the client software comprises computer readable instructions which, when executed by a client computer, cause the client computer to provide to a computer server over a computer network a respective index at least upon changes to any patient record in a respective database;
receiving, by a computer server and over the computer network, indexes provided by the client software installed on at least some the plurality of geographically disparate database systems, wherein each index comprises data encoding at least one patient and at least one associated clinical fact stored at a respective database;
merging, by the computer server, data from the indexes provided by the client software installed on each of the plurality of geographically disparate database systems to produce a master index;
identifying a plurality of patients that have upcoming appointments or recent changes to their clinical facts;
for each of the plurality of patients, building in transient electronic memory a respective representation of encoded clinical facts from multiple of the plurality of geographically disparate database computer systems using the master index;
comparing each respective representation of encoded clinical facts to each of a plurality of clinical event templates to detect at least one match; and
for at least one match, providing a notification to a care provider of a respective patient.

2. The method of claim 1, wherein no clinical records leave their respective database of the plurality of disparate database computer systems.

3. The method of claim 1, wherein the encoded clinical facts comprise encrypted clinical facts.

4. The method of claim 1, wherein the plurality of disparate database computer systems comprise a plurality of geographically disparate database computer systems, a plurality of legally disparate database computer systems, or a plurality of technologically disparate computer systems.

5. The method of claim 1, wherein the identifying comprises:
identifying by at least one of the plurality of disparate database computer systems; and
providing corresponding identifying data to the computer server.

6. The method of claim 5, further comprising providing, by the computer server, the master index to at least one of the plurality of disparate database computer systems.

7. The method of claim 6, wherein the building in transient electronic memory a respective representation of encoded clinical facts is performed by least one of the plurality of disparate database computer systems.

8. The method of claim 1, wherein the client software further comprises computer readable instructions which, when executed by a client computer, cause the client computer to translate changes to patient records into encoded clinical facts.

9. The method of claim 1, wherein the notification comprises at least one electronic link to a clinical record of a respective patient in a respective one of the plurality of geographically disparate database computer systems.

10. The method of claim 9, further comprising, prior to the providing a notification, receiving authorization from the respective one of the plurality of geographically disparate database computer systems to provide the link.

11. A computer system comprising:
non-transitory computer-executable client software installed on a plurality of disparate database computer systems communicatively coupled to a computer network, wherein the client software comprises computer readable instructions which, when executed by a respective client computer, cause the respective client computer to provide to a computer server a respective index at least upon changes to any patient record in a respective database; and
a computer server computer communicatively coupled to the computer network, wherein the computer server is configured to:
receive indexes provided by the client software installed on at least some of the plurality of geographically disparate database systems, wherein each index comprises data encoding at least one patient and at least one associated clinical fact stored at a respective database, and
merge data from the indexes provided by the client software installed on each of the plurality of geographically disparate database systems to produce a master index;
wherein the system is further configured to:
identify a plurality of patients that have upcoming appointments or recent changes to their clinical facts;
for each of the plurality of patients, build in transient electronic memory a respective representation of encoded clinical facts from multiple of the plurality of geographically disparate database computer systems using the master index;
compare each respective representation of encoded clinical facts to each of a plurality of clinical event templates to detect at least one match; and
for at least one match, provide a notification to a care provider of a respective patient.

12. The system of claim 11, wherein no clinical records leave their respective database of the plurality of disparate database computer systems during operation of the system.

13. The system of claim 11, wherein the encoded clinical facts comprise encrypted clinical facts.

14. The system of claim 11, wherein the plurality of disparate database computer systems comprise a plurality of geographically disparate database computer systems, a plurality of legally disparate database computer systems, or a plurality of technologically disparate computer systems.

15. The system of claim 11, wherein the system is further configured to identify a plurality of patients that have upcoming appointments or recent changes to their clinical facts by:
identifying, by at least one of the plurality of disparate database computer systems, a plurality of patients that have upcoming appointments or recent changes to their clinical facts; and
providing, by at least one of the plurality of disparate database computer systems, corresponding identifying data to the computer server.

16. The system of claim 15, wherein the computer server is further configured to provide the master index to at least one of the plurality of disparate database computer systems.

17. The system of claim 16, wherein the system is further configured to build in transient electronic memory of at least one of the plurality of disparate database computer systems a respective representation of encoded clinical facts.

18. The system of claim 11, wherein the client software further comprises computer readable instructions which, when executed by a client computer, cause the client computer to translate changes to patient records into encoded clinical facts.

19. The system of claim 11, the notification comprises at least one electronic link to a clinical record of a respective patient in a respective one of the plurality of geographically disparate database computer systems.

20. The system of claim 19, wherein the system is further configured to, prior to providing a notification, receive authorization from the respective one of the plurality of geographically disparate database computer systems to provide the link.
